(19) United States
(12) Patent Application Publication
(10) Pub. No.: US 2007/0042020 A1
(43) Pub. Date: Feb. 22, 2007

(54) Composition for the treatment of dysfunctional energy metabolism syndrome

(76) Inventor: James R. Howard, Brawley, CA (US)

Correspondence Address: THE WEBB LAW FIRM, P.C., 700 KOPPERS BUILDING, 436 SEVENTH AVENUE, PITTSBURGH, PA, 15219, US

(21) Appl. No.: 10/562,517

(22) PCT Filed: Oct. 29, 2004
(86) PCT No.: PCT/US04/36308

§ 371 Date: Jun. 30, 2006

Related U.S. Application Data
(60) Provisional application No. 60/515,796, filed on Oct. 30, 2003.

(51) Int. Cl. A61K 31/455; A61K 31/22; A61K 31/205; A61K 31/198; A61K 47/00

(52) U.S. Cl. 424/439; 514/356; 514/561; 514/546; 514/554

(57) ABSTRACT

A nutritional composition for the treatment of syndromes related to dysfunctional energy metabolism is provided. The composition comprises a combination of L-carnitine, acetyl-L-carnitine, pantothenate and niacinamide. Methods of treatment of dysfunctional energy metabolism are also disclosed.

### BACKGROUND OF THE INVENTION

1. Field of the Invention

The present invention relates to treatment of humans and other mammals suffering from syndromes related to dysfunctional energy metabolism that stand alone or complicate diseases related to nuclear genome mutations and, in particular, but not limited to, seizuring and cardiomyopathy. The treatment procedure supplements cofactors to enzymes functional in energy metabolism pathways that are contributing to clinical disease. More specifically, the treatment comprises administration of L-carnitine, acetyl-L-carnitine, pantothenate and niacinamide.

2. Description of Related Art

Most cellular energy production is carried on by mitochondria. Dysfunctional energy metabolism by defective mitochondria results when mutated, inefficient enzymes involved in energy metabolism exist; when cofactors to energy metabolism enzymes are deficient or are inactivated; and when abnormal frameworks exist within mitochondria that mal-position enzymes, thus impairing their contribution to energy production. In people, nearly 500 nuclear genome mutations have been associated with defective mitochondria and nearly 100 mutations of mitochondrial DNA have been described. Not all DNA mutations cause measurable decline in oxidative phosphorylation, the last stage of energy production. This is not to say associated clinical syndromes do not exist or that these syndromes cannot be affected by improved energy metabolism. Cofactor deficiencies may relate to impaired enteral absorption, excessive renal excretion, diminished availability in diet, and reduced synthesis or conservation. Also, cofactor-responsive syndromes have been encountered in the face of excessive amounts of cofactors in serum that appear to have been metabolically tied up or otherwise inactivated.

Clinical syndromes are the consequence of impaired function of anomalous metabolic pathways in one or more organs. Thus, onset of syndromes, organ involvement, and progression of symptoms may vary between cases. Dysfunctional energy metabolism may also potentiate onset and contribute to severity of many heritable diseases. There exist rules of thumb suggestive of mitochondrial diseases: A common disease develops that has unusual features setting it apart from the typical disease, a syndrome may involve several organs, and recrudescence of a chronic disease is initiated by an infection or other stressful event. Currently, diagnosis of dysfunctional energy metabolism is based on mitochondrial DNA analysis, blood and cerebrospinal fluid lactate and pyruvate content or brain magnetic resonance spectroscopy, urine organic acid analysis, plasma and urine amino acid content, blood and urine carnitine, brain magnetic resonance, and muscle biopsy. These tests, while definitive, are expensive and usually conducted in a few specialty centers. They are not available to animal medicine, to people with limited economic means, and in areas or countries where the technology does not exist or is beyond the economic reach of patients. Another way to identify health conditions associated with defective energy metabolism is to provide cofactors: vitamins, vitamin-like compounds, and trace minerals associated with energy production, and monitor patient responses to them.

In theory, energy production is a continuum involving five segments where enzyme defects or cofactor deficits can impair function that are amenable to treatment with physiological amounts of related cofactors. They are: (1) translocation of nutritional substrates into mitochondria by actions of L-carnitine and acetyl-L-carnitine; (2) transfer of acetyl groups emanating from metabolic oxidation of the translocated substrates to the citric acid cycle with the aid of pantothenate in coenzyme A; (3) metabolism of the acetyl groups to provide free electrons in the citric acid cycle with the aid of flavin coenzymes; (4) transport of electrons to the electron transport chain by niacinamide and its derivatives; and (5) oxidative phosphorylation of ADP to ATP with catalytic assistance of ubiquinone.

Provision of pertinent enzyme cofactors has been reported to potentiate synthesis of related enzymes. U.S. Pat. Nos. 5,889,055 and 5,973,027 teach the relevance of these theoretical considerations and demonstrate there is variability among patients and that multiple tissues with varied dominance of pathways of energy production are commonly involved in patients with dysfunctional energy metabolism. U.S. Pat. No. 6,562,869 also discloses theoretical considerations and a nutritional supplement containing carnitine in combination with an anti-oxidant, a carbohydrate source and at least one B vitamin.

### SUMMARY OF THE INVENTION

Accordingly, the present invention provides a nutritional composition comprising L-carnitine, acetyl-L-carnitine, pantothenate, and niacinamide. Preferably, these compounds are combined into an aqueous solution for oral administration on a daily basis or injected for limited periods of treatment. The combination of ingredients can also be mixed into the diet. The invention enhances energy metabolism and is useful for prevention or treatment of syndromes related to energy deficit associated with dysfunctional energy metabolism.

### DETAILED DESCRIPTION OF THE INVENTION

In one aspect, the present invention provides a nutritional composition adapted for supplementing cofactors of enzymes involved with energy production by humans and other mammals, the composition comprising L-carnitine, acetyl-L-carnitine, pantothenate and niacinamide. Preferably, the composition is provided in the form of an aqueous solution, although other dosage forms such as powder, crystal, tablet, gel caps, concentrates and the like are also contemplated. Administration can be direct oral, parenteral, or by other routes known to one skilled in the art.

The nutritional composition of the present invention facilitates energy production of cells and tissues thereby enhancing skeletal muscle activity, cardiac function, renal tubule and hepatocyte synthetic and detoxification functions, mental acuity, and spermatozoal vitality and viability. Summarily, it enhances function of organs plagued by energy deficits related to impaired energy production.

In the aqueous dosage form, the following amounts are used, in milligrams per milliliter of aqueous solution: L-carnitine 0.0005 to 625, acetyl-L-carnitine 0.0005 to 625, pantothenate 0.000005 to 10 and niacinamide 0.000005 to 20, assuming a consumption range of about 1-5 ml/kg body weight. The preferred range of ingredients for direct oral or parenteral application in milligrams per milliliter final solution is: 50 to 150 L-carnitine, 50 to 150 acetyl-:-carnitine, 0.5 to 5 calcium pantothenate, and 0.5 to 5 niacinamide. A preferred range of ingredients for soft or sport drinks in milligrams per milliliter is: 0.05 to 3 L-carnitine, 0.05 to 3 acetyl-L-carnitine, 0.0005 to 0.003 pantothenate, and 0.0005 to 0.003 niacinamide. Range of ingredients for semen extending solution in milligrams per milliliter is: 0.0005 to 0.03 L-carnitine, 0.0005 to 0.03 acetyl-L-carnitine, 0.000005 to 0.00003 calcium pantothenate and 0.000005 to 0.00003 niacinamide. The preferred range of ingredients for concentrated and super-saturated solutions for addition to soft drinks or encapsulation into gel caps is: 150 to 625 L-carnitine, 150 to 625 acetyl-L-carnitine, 5 to 20 calcium pantothenate, and 5 to 20 niacinamide. In general, the four ingredients will be combined in the following ratio: L-carnitine:acetyl-L-carnitine:pantothenate:niacinamide as about 1:0.1-2:0.01-0.5:0.01-0.5.

In another aspect, the present invention provides a method of treating dysfunctional energy metabolism in a mammal comprising administering an effective amount of a composition comprising L-carnitine, acetyl-L-carnitine, pantothenate and niacinamide to a mammal in need of such treatment. As used herein, the term “effective amount” refers to the amount of the combination of compounds necessary to alleviate or reduce the symptoms of dysfunction energy metabolism. Typically, an effective amount will be about 0.1-20 mg/kg body weight L-carnitine, about 0.1-20 mg/kg body weight acetyl-L-carnitine, about 0.1-1.0 mg/kg body weight pantothenate and about 0.1-1.0 mg/kg body weight niacinamide per day. This treatment can be carried out on a daily basis for a period of months or years, if necessary.

As used herein, the term “acetyl-L-carnitine” will refer to the compound itself and other closely related compounds such as propyl-L-carnitine, as are known by one skilled in the art. Also as used herein, the term “pantothenate” will refer to pantothenate, also known as Vitamin B5, and other commonly known and related forms of this compound, including by way of example, pantothenic acid, sodium pantothenate, and calcium pantothenate. As used herein, the term “niacinamide” will refer to the compound itself, also known as Vitamin B3 and other known forms such as niacin and nicotinic acid.

Compositions of the present invention preferably include a preservative. Suitable preservatives for addition to these preparations on weight per volume basis are: about 0.1 percent methylparaben; about 0.1 percent methylparaben with about 0.03 percent propylparben sodium; about 0.07 percent potassium sorbate with about 0.07 percent sodium benzoate; and about 0.5 percent benzoic acid.

The term “cofactor” refers to L-carnitine, acetyl-L-carnitine, pantothenate, and niacinamide compounds which conjoin with their related enzymes of energy metabolism in mammalian cells. “Parenteral” shall mean any administrative mode other than oral enteral and shall include subcutaneous, intramuscular, and intravenous injection.

“Syndromes” is a symptom, or complex of symptoms occurring together, often with associated biomedical anomalies, which may characterize a specific disease entity or complicate a specific disease entity. “Dysfunctional energy metabolism” is a condition in which production of adenosine triphosphate by mitochondria of one or more body tissues is impaired to such an extent that a syndrome can ensue. “Cofactor responsive syndrome or disease” represents a disease, syndrome, or syndrome complex benefited by amelioration of signs and/or symptoms toward the normal through treatment of a patient with combined L-carnitine, acetyl-L-carnitine, pantothenic acid, and niacinamide. Some examples of relevant syndromes are: muscle weakness, chronic pain, myocardial insufficiency, seizuring, reduced mental function, and lipemia. The purpose of the invention is to treat or prevent the development of dysfunctional energy metabolism syndromes related to increased need of, or due to inadequate intake, synthesis, or conservation of one or more of the following cofactors L-carnitine, acetyl-L-carnitine, pantothenate, and/or niacinamide.

Clinical studies demonstrate the interrelationship of cofactors: One old Labrador Retriever dog that had difficulty in standing and walking was able to increase the distance it could walk from 340 yards to 1240 yards after one week of L-carnitine supplementation at 5 mg/kg body weight, but after two weeks of supplementation with acetyl-L-carnitine at 4 mg/kg body weight and no L-carnitine exercise, ability had declined to 648 yards. When L-carnitine, acetyl-L-carnitine, with added pantothenate at 0.4 mg/kg body weight were supplemented for one week, exercise ability increased to 2740 yards and, by three weeks, ability was 3807 yards. However, when pantothenate was eliminated for two weeks, exercise ability declined to 350 yards. Then when pantothenate was returned to the L-carnitine and acetyl-L-carnitine combination, his exercise capability returned to more than 3800 yards. This case illustrates the contribution of the various cofactors to physical activity.

However, syndromes may develop that are non-responsive to the L-carnitine, acetyl-L-carnitine and pantothenic acid combination. Such was the case with an eleven-year-old terrier-poodle crossbred dog presented with heart failure. She was treated with the combination of three cofactors, L-carnitine, acetyl-L-carnitine, and pantothenate. Her heart condition improved and she was without symptoms for three months. She was again presented in a state near death. She was salivating profusely and the owner stated the dog was unable to eat her nutritionally-balanced commercial diet. Examination revealed the anterior half of the tongue was necrotic and nonfunctional. Her heart was okay but the tongue's condition was diagnosed as pellagra, black tongue. With the plethora of readily available, well-formulated pet foods, this is a very rare condition in America today. It is associated with deficiency of niacin. In spite of sloughing the necrotic portion of its tongue, with niacinamide added to the three-cofactor combination daily supplementation, the dog promptly recovered. This case revealed an unintended need for niacin or its derivatives in combinations of cofactors for enhancement of energy metabolism. Further studies were done with different syndromes as exemplified in the detailed description of the invention. The invention provides practicable, convenient, and economical means of providing L-carnitine, acetyl-L-carnitine, pantothenate, and niacinamide for recognizing, preventing and treating syndromes associated with dysfunctional energy metabolism.

### EXAMPLE 1

This example pertains to a preferred, stable aqueous solution containing per mL: 100 mg L-carnitine, 80 mg acetyl-L-carnitine, 2 mg calcium pantothenate, and 4 mg niacinamide. One liter of the solution is prepared at room temperature by dissolving 100 grams of L-carnitine in 700 mL deionized water. In this solution 80 grams of acetyl-L-carnitine are dissolved, followed by 2 grams of calcium pantothenate and 4 grams of niacinamide. One gram of methylparaben is added and dissolved as preservative. Sufficient water is added to bring the final quantity to 1000 mL. This final solution is filtered to remove bacteria and like-sized microbes and is bottled in sterile, amber-glass or plastic. pH of the solution is about 4. Its tart, mildly acidic taste enhances many food flavors and is intended for direct daily oral administration at about 1 mL per 20 kilogram body weight or it can be added daily to diet or drinks to provide 5 mg L-carnitine, 4 mg acetyl-L-carnitine, 0.5 mg pantothenate and 1 mg niacinamide per kilogram of body weight. The preferred product can be used as an aid to treat heart failure on a long-term basis and improve exercise tolerance. Coughing related to pulmonary edema is diminished or eliminated, and life is prolonged. Some dogs with adult-onset seizuring or behavior changes have been returned to normal when their diets have been supplemented with this preparation. Patients with chronic pain similar to fibromyalgia and/or muscle weakness suggestive of chronic fatigue syndrome have improvement of symptoms when consuming this preparation.

### EXAMPLE 2

This example pertains to preparation of one liter of sterile, neutral, stable aqueous solution containing per mL: 50 mg L-carnitine, 40 mg acetyl-L-carnitine, 1 mg calcium pantothenate, and 2 mg niacinamide. A solution is prepared at room temperature by dissolving 50 grams of L-carnitine in 700 mL pyrogen-free distilled water. When that is dissolved, 40 grams of acetyl-L-carnitine is added and dissolved, followed by 1 gram calcium pantothenate and 2 grams of niacinamide. The pH of the solution is adjusted to pH 7 with normal NaOH, water is added, bringing the final volume to 1000 mL, and it is filtered to remove microbes and like-sized microbes. It is then aliquoted in sterile 10 mL amber-glass vials and sealed.

The solution is intended for subcutaneous, intramuscular, or intravenous injection at the rate of about 1 milliliter per 20 kilogram body weight. It can be injected directly or admixed into intravenous electrolyte and glucose solutions administered to hospitalized patients. This material has been used to successfully treat patients with acute cardiac insufficiency and shock. It has also been injected intravenously into patients with cardiac insufficiency prior to surgery to prevent heart failure during surgery, and it has also been injected into patients shortly after developing symptoms of stroke and/or brain thrombo-embolism to improve mental function.

### EXAMPLE 3

This example pertains to the addition of the cofactor combination to soft drinks, health drinks, fruit punches, or sports drinks with a range of dissolved cofactors in milligrams per milliliter of finished product of 0.5 to 2 mg L-carnitine, 0.5 to 2 mg acetyl-L-carnitine, 0.01 to 0.4 mg pantothenate, and 0.02 to 0.4 mg niacinamide.

### EXAMPLE 4

The individual ingredients of the preparation can be added as powders or crystals to prepared diets at time of manufacture or time of consumption in the following ratios relative to L-carnitine: acetyl-L-carnitine 0.1 to 2; pantothenate 0.01 to 0.5; and niacinamide 0.01 to 0.5; in amounts to provide the consumer with L-carnitine 0.1 to 20 mg per kilogram body weight; acetyl-L-carnitine 0.1 to 20 mg per kilogram body weight; pantothenate 0.01 to 1 mg per kilogram body weight; and niacinamide 0.01 to 1 mg per kilogram body weight.

### EXAMPLE 5

This preparation is useful in the preferred preparation in EXAMPLE 1 as an aid to enhancing spermatozoal motility when consumed by males when spermatozoal hypomotility is a problem and for enhancing spermatozoal viability and vitality where semen storage, processing and freezing are practiced. Also, the preparation is useful where the latter procedures are in practice when it is added to semen diluents to enhance spermatozoal viability and vitality in concentration ranges of micrograms per milliliter of 1 to 100 L-carnitine; 1 to 100 acetyl-L-carnitine; 0.02 to 2 pantothenate; and 0.02 to 4 niacinamide.

### EXAMPLE 6

This example pertains to the preparation of an alternative mode of therapy through concentrate and super-saturated solutions of ingredients that are encapsulated and consumed in pill form. Said pills may contain a dosage range of ingredients in milligrams per milliliter: L-carnitine 100 to 625; acetyl-L-carnitine 100 to 625; pantothenate 2 to 10; and niacinamide 2 to 10.

To prepare one liter of a preferred super-saturated solution: Combine and mix thoroughly 500 grams of powdered or crystalline L-carnitine and 400 grams of powdered or crystalline acetyl-L-carnitine. Dissolve 10 grams of calcium pantothenate and 20 grams of niacinamide in 90 mL water; add this solution to the L-carnitine, acetyl-L-carnitine mixture and stir until thoroughly mixed. Microwave the mixture to 80 to 90° C. and continue to stir. Fine gas bubbles will form. Maintain an elevated temperature of about 70° as bubbles rise to the top and escape. Placing the warmed mixture in a vacuum will facilitate this process. As the gas is removed, the mixture becomes a clear, transparent, viscid fluid. Cool the solution to room temperature and stir in additional water to bring the volume to one liter. The small amount of water in the pantothenate-niacinamide solution facilitates binding of L-carnitine and acetyl-L-carnitine within a few hours rather than weeks or months as happens in the absence of water. Microwaving the mix further facilitates binding in a matter of minutes instead of hours. A hot plate or other heating device can be used in place of microwave, but liquefaction of the mixture is slower. Clearing is enhanced by placing the solution in a vacuum so the bubbles escape from the surface of the solution.

It is thought that the formed gas is hydrogen, liberated as covalent bonds form between L-carnitine and acetyl-L-carnitine. Preliminary study shows this reaction is not deleterious to the preparation's physiological activity: A person who had been consuming the preferred preparation in EXAMPLE 1 substituted the super-saturated solution for one month with no evidence of diminished energy nor detrimental effects.

The final super-saturated solution has a pH of about 7 and density greater than water, one milliliter weighs about 1225 milligrams. On dilution of the super-saturated solution pH declines to about 3.85. The high osmoliarity of the final super-saturated solution may inhibit microbial growth. In one study inoculation with multiple bacterial strains produced no observable growth after six months of incubation. The final solution can be placed in soft gel capsules for oral administration. Concentrated and super-saturated solutions can be packaged in bulk for distribution or storage purposes to be diluted into semen dilution solution, soft and other drinks, and therapeutic preparations.

### EXAMPLE 7

This example provides a description of the preferred preparation in a dog with brain involvement. A five-year-old, spayed Maltese/Pomeranian bitch began having grand mal seizures once a month. Seizures would endure for a few seconds to as long as ten minutes. Seizure frequency increased during the ensuing year until she was having one or two seizures a week. To control the seizures, combined phenobarbitol and KBr solution administered but they were only partially successful and seizures continued. Her medication was changed to the preferred solution minus niacinamide with anti-convulsants added and he seizuring was controlled but when anti-convulsants were discontinued seizuring resumed. The cofactor solution was replaced by the preferred solution and convulsions ceased. When the anti-convulsants were discontinued three months later, there were no further seizures, as long as the combination of cofactors was administered every day.

### EXAMPLE 8

This example demonstrates use of the preferred preparation of Example 1. A ten-year-old boy diagnosed with phenylketonuria and mitochondrial disease associated with low tissue carnitine was treated for four years with L-carnitine, acetyl-L-carnitine, and pantothenate once daily. Periodically, he would fall asleep in school following stressful exercise or examinations. It was thought his sleeping might be due to exhaustion related to defects in the citric acid cycle or oxidative phosphorylation. To evaluate this, he began consuming the preferred preparation. This change resulted in his no longer falling asleep in school and his level of playtime activity has increased.

Whereas particular embodiments of this invention have been described above for purposes of illustration, it will be evident to those skilled in the art that numerous variations of the details of the present invention may be made without departing from the scope of the invention as defined in the appended claims.

The invention claimed is:

1. A nutritional composition for treatment of impaired or inadequate energy production or dysfunctional energy metabolism in a mammal comprising L-carnitine, acetyl-L-carnitine, pantothenate and niacinamide.

2. The composition of claim 1, wherein said L-carnitine, acetyl-L-carnitine, pantothenate and niacinamide are present in said composition in a ratio of about 1:0.1-2:0.01-0.5:0.01-0.5.

3. The nutritional composition of claim 1, further comprising a preservative.

4. The nutritional composition of claim 1, in an aqueous solution.

5. The nutritional composition of claim 4, wherein the concentration of each ingredient in the aqueous solution is: L-carnitine 0.0005 to 500 mg/ml, acetyl-L-carnitine 0.0005 to 500 mg/ml, pantothenate 0.000005 to 10 mg/ml and niacinamide 0.000005 to 20 mg/ml.

6. The nutritional composition of claim 1, in a concentrate.

7. The nutritional composition of claim 1, in a soft drink.

8. The nutritional composition of claim 1, in a gel cap.

9. A nutritional composition for treatment of impaired or inadequate energy production or dysfunctional energy metabolism in a mammal consisting essentially of L-carnitine, acetyl-L-carnitine, pantothenate and niacinamide.

10. A method of treating impaired or inadequate energy metabolism or dysfunctional energy metabolism in a mammal comprising administering an effective amount of a composition comprising L-carnitine, acetyl-L-carnitine, pantothenate and niacinamide to a mammal in need of such treatment.

11. The method of claim 10, wherein said composition consists essentially of L-carnitine, acetyl-L-carnitine, pantothenate and niacinamide.

12. The method of claim 10, wherein said composition further comprises a preservative.

13. The method of claim 11, wherein said composition further comprises a preservative.

14. The method of claim 10, wherein said dysfunctional energy metabolism comprises cardiomyopathy.

15. The method of claim 10, wherein said dysfunctional energy metabolism comprises seizures.

16. The method of claim 10, wherein said administration is oral.

17. The method of claim 10, wherein said administration is parenteral.

18. The method of claim 10, wherein said effective amount is about 0.1-20 mg/kg L-carnitine, about 0.01-20 mg/kg acetyl-L-carnitine, about 0.01-1.0 mg/kg pantothenate and about 0.01-1.0 mg/kg niacinamide per day.
